(19) United States
(12) Patent Application Publication
(10) Pub. No.: US 2024/0299287 A1
(43) Pub. Date: Sep. 12, 2024

(54) Make-up Composition with Retention of the Matte Effect

(71) Applicant: L V M H Recherche, Saint-Jean De Braye (FR)

(72) Inventors: Elodie Champredonde, Saint-Jean De Braye (FR); Valérie Bouchard De La Poterie, Saint-Jean De Braye (FR)

(21) Appl. No.: 18/268,727

(22) PCT Filed: Dec. 23, 2021
(86) PCT No.: PCT/FR2021/052441

§ 371 Date: Jun. 21, 2023

(30) Foreign Application Priority Data
Dec. 23, 2020 (FR) 2014057

(51) Int. Cl. A61K 8/898; A61K 8/25; A61K 8/31; A61Q 1/06

(52) U.S. Cl. CPC A61K 8/898; A61K 8/25; A61K 8/31; A61Q 1/06; A61K 2800/31; A61K 2800/43; A61K 2800/651

(57) ABSTRACT

The present invention relates to a cosmetic composition for care and/or make-up of keratinous materials, in particular the lips, comprising, in a physiologically acceptable medium: a) a silicone/polyurethane polymer with an active material content of at least 17.5% by weight relative to the total weight of the composition, b) one or more linear or branched C8-C19 alkanes, c) a silica powder, preferably a processed hydrophobic powder, and d) optionally pigments.

### FIELD OF THE INVENTION

The present invention relates to the cosmetics field and in particular to cosmetic compositions for make-up of keratinous materials, in particular the lips.

### STATE OF THE ART

Lip care and/or make-up products intended to beautify, protect and/or color the lips are widely used. In particular, products are known for the lips, in liquid, semi-fluid or solid (stick) form, for a glossy deposit or a matte deposit on the lips and having, for make-up products, good color-lasting properties. To provide the matte effect, volatile oils and fillers are generally used, but the stability of the composition may be impacted and the deposit of the film may have a drying appearance on the lips.

Therefore, the need to develop new care and/or make-up products for the lips, in particular liquid products, for obtaining a homogeneous, comfortable, matte deposit on the lips, like a second skin, still remains.

The present invention allows to meet these needs. The Applicant has in fact developed a composition comprising a silicone/polyurethane polymer with an active material content of at least 17.5% by weight relative to the total weight of the composition, one or more linear or branched C8-C19 alkanes, and a silica powder, preferably a hydrophobic treated powder. Unexpectedly, the Applicant has demonstrated that it is possible to formulate a composition comprising a content of active material of silicone/polyurethane polymer in excess of 16% by weight, without generating resistance to friction or loss of lasting, even in the absence of a silicone resin associated with the silicone/polyurethane polymer, contrary to the prejudices taught in application FR3052357. The composition of the invention even has an unequaled matte performance and matt lasting compared with the matte fluid products for the lips present on the market. More particularly, the Applicant has shown that a composition according to the invention applied as a film to the lips does not transfer when it contacts a fabric or a material on which it is applied. In addition to the particular make-up effect conferred by the silicone polyurethane polymer combined with the other ingredients in the composition of the invention, this transfer resistance property is particularly sought for users who seek to avoid any alteration of the colored film by contact with a support, material or fabric.

### DISCLOSURE OF THE INVENTION

A first aspect of the invention is therefore a cosmetic composition for care and/or make-up of keratinous materials, in particular the lips, comprising, in a physiologically acceptable medium:
a) A silicone/polyurethane polymer with an active material (dry material) content of at least 17.5% by weight relative to the total weight of the composition,
b) One or more linear or branched C8-C19 alkanes,
c) A silica powder, preferably a hydrophobic treated powder, and
d) Optionally pigments.

By ‘keratinous materials’ is meant in particular the skin and/or the lips, the hair, the eyelashes and the nails. According to a particular embodiment, the composition of the invention is applied to the lips.

The invention also relates to a cosmetic method for caring for and/or making up keratinous materials, in particular the lips, comprising the application to said keratinous materials, in particular the lips, of a composition according to the invention.

The application of the composition of the invention allows the deposition on keratinous materials and in particular the lips, of a matte, homogeneous and comfortable film, like a second skin.

### DETAILED DESCRIPTION OF THE INVENTION

Thus, a first aspect of the invention is a cosmetic composition for care and/or make-up of keratinous materials, in particular the lips, comprising, in a physiologically acceptable medium:
a) A silicone/polyurethane polymer with an active material (dry material) content of at least 17.5% by weight relative to the total weight of the composition,
b) One or more linear or branched C8-C19 alkanes,
c) A silica powder, preferably a hydrophobic treated powder, and
d) Optionally pigments

According to a particular and preferred embodiment, the composition of the invention is an anhydrous composition.

The term “anhydrous” means in particular that the water is preferably not added to the compositions but may be present in trace form in the various compounds used in the compositions. In particular, the composition according to the invention comprises less than 4% by weight of water, preferably less than 3%, preferably less than 2%, more preferably less than 1%, even more preferably less than 0.5% by weight of water, relative to the total weight of said composition, or even is completely free of water.

According to a particular and preferred embodiment, the composition of the invention is a liquid and anhydrous composition.

The composition of the invention therefore comprises a silicone/polyurethane polymer as defined below with an active material (dry material) content of at least 17.5% by weight relative to the total weight of the composition.

### Silicone/Polyurethane Polymer

By “silicone/polyurethane polymer” according to the invention, is meant any polymer comprising organosiloxane units and urethane bonds.

According to a particular embodiment, the silicone/polyurethane polymer according to the invention is the reaction product of a polyorganosiloxane functionalized with hydroxyl groups, preferably comprising at least two hydroxyl groups, with a diisocyanate compound.

Such a polyorganosiloxane functionalized with hydroxyl groups corresponds to the formula formula (I)

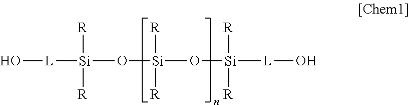

wherein R is selected independently at each occurrence from a hydrogen atom, a hydroxyl group, and optionally substituted hydrocarbon groups containing from 1 to 10 carbon atoms, and in particular from an alkyl, alkenyl, alkynyl, aryl, aryl-alkyl or alkyl-aryl group which are substituted or not; preferably, R is selected from linear, cyclic or branched and optionally substituted C1-C6 alkyl or alkenyl groups, including, without limitation, methyl, ethyl, propyl, isopropyl, butyl, isobutyl, t-butyl, amyl, hexyl, cyclohexyl, vinyl, C1-C8 allyl or aryl, aryl-alkyl or alkyl-aryl groups, including, without limitation, phenyl, benzyl, tolyl, xylyl,
wherein each of the above groups R may be optionally substituted with one or more heteroatoms, including oxygen, nitrogen, phosphorus and halogen, in particular fluorine, as illustrated by fluoroalkyl groups (perfluoroalkyls) such as mono-, di-, and tri-fluoromethyl, perfluorophenyl, and C1-C6 substituted alkyl amino groups, including those having the formula —(CH₂)₁₋₆—NR^{N}₂ and —(CH₂)₁₋₆—NR^{N}—(CH₂)₁₋₆—NR^{N}₂ where R^{N} is typically hydrogen, but can also be methyl, ethyl, propyl, and the like; polyether groups, including, without limitation, polyethylene oxide groups having the formula —(CH₂CH₂O)ₙ—, propylene oxide groups having the formula —(CH(CH₃)CH₂O)ₙ— and combinations thereof; and amine oxide, phosphate, hydroxyl, ester and/or carboxylate functions, or the like; or
wherein R may comprise an additional -L-OH group;
   wherein L represents either a bond or a bonding group; preferably L is a bonding group selected from divalent hydrocarbons having 1 to 10 carbon atoms, including a divalent alkyl, alkenyl, alkynyl, aryl, alkyl-aryl or aryl-alkyl group, such as for example a C1-C10 alkyl group, including, without limitation, divalent groups having the formula —(CH₂)₁₋₁₀—, preferably —(CH₂)₁₋₆—, and more preferably, L is —CH₂CH₂CH₂—;
and where n is an integer between 0 and 5000, preferably between 1 and 200, more preferably between 10 and 100, and even more preferably between 10 and 50.

Preferably, R represents at least one or more occurrences of a methyl group, more preferably, R represents a methyl group at all or almost all of the occurrences, which means that R represents a methyl group at more than 90% of occurrences, in particular at more than 95% of occurrences, or even at more than 98% of occurrences. According to one embodiment of the invention, the polyorganosiloxane functionalized with hydroxyl groups comprises a polymethylsiloxane corresponding for example to the structure of formula Ia:

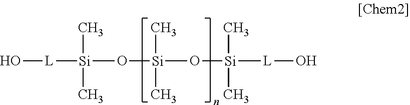

wherein L and n are as previously defined.

According to a preferred embodiment of the invention, the polyorganosiloxane functionalized with hydroxyl groups comprises a polymethylsiloxane corresponding for example to the structure of formula Ib:

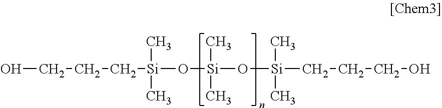

where n is as defined above.

The diisocyanate may in particular have the formula O═C═NR¹—N═C═O, where R¹ is a divalent hydrocarbon group containing from 1 to 20 carbon atoms, including 20 optionally substituted by one or more heteroatoms, and in particular R¹ can be selected from a linear, cyclic or branched, optionally substituted cyclic alkyl, alkenyl, alkynyl, aryl, alkyl-aryl or arylalkyl group, including, without limitation:
i. a group having the formula:

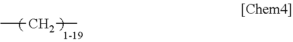

ii. a group having the formula:

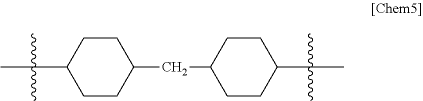 [SMILES: *C1CCC(CC2CCC(*)CC2)CC1]

iii. a group having the formula:

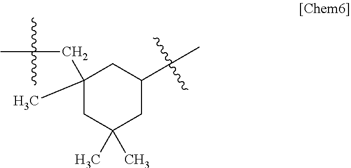 [SMILES: *CC1(C)CC(*)CC(C)(C)C1]

iv. a group having the formula:

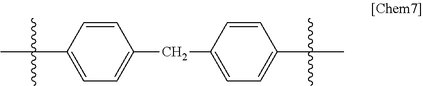 [SMILES: *c1ccc(Cc2ccc(*)cc2)cc1]

v. a group having the formula:

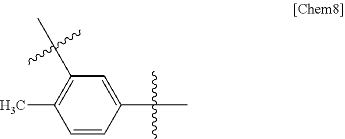 [SMILES: *c1ccc(C)c(*)c1]

and combinations thereof.

Diisocyanates that may be suitable within the scope of the invention comprise, without limitation, toluene diisocyanate; methylene diphenyl diisocyanate, including 2,2′-MDI, 2,4′-MDI, and 4,4′-MDI; 1,6-hexamethylene diisocyanate; isophorone diisocyanate; dicyclohexylmethane 4,4′-diisocyanate; xylene diisocyanate; cyclohexane diisocyanate; 3,3′-dimethyl-4,4′-diphenylmethane diisocyanate; p-phenylene diisocyanate; m-phenylene diisocyanate; 4,4′-isopropylidene dicyclohexyl isocyanate; and their equivalents.

In a preferred embodiment, the diisocyanate is selected from the group consisting of 1,6-hexamethylene diisocyanate, dicyclohexylmethane 4,4′-diisocyanate, isophorone diisocyanate, and combinations thereof.

In one embodiment, the diisocyanate comprises or consists essentially of 1,6-hexamethylene diisocyanate.

In another embodiment, the diisocyanate comprises or consists essentially of isophorone diisocyanate.

In yet another embodiment, the diisocyanate comprises or consists essentially of dicyclohexylmethane 4,4′-diisocyanate, this embodiment is particularly preferred.

Thus, according to a particular embodiment, the silicone/polyurethane polymer is the reaction product
of one polyorganopolysiloxane functionalized with hydroxyl groups, in particular corresponding to the structure of formula (I)

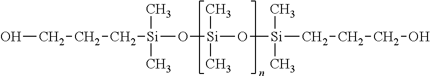

wherein n is an integer between 0 and 5000, preferably between 1 and 200, more preferably between 10 and 100, and even more preferably between 10 and 50,
with a diisocyanate compound, in particular selected from the group consisting of 1,6-hexamethylene diisocyanate, dicyclohexylmethane 4,4′-diisocyanate, isophorone diisocyanate, and combinations thereof.

According to a preferred embodiment, the silicone/polyurethane polymer according to the invention comprises recurring units derived from the polyorganosiloxane functionalized with hydroxyl groups and from the diisocyanate in the form of an alternating copolymer AB, where the unit A has the structure of formula II:

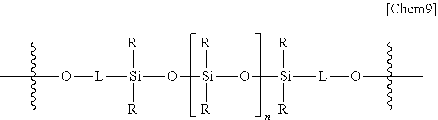

wherein R, L and n are defined as above in relation to formula I, Ia, Ib and Ie, and wherein unit B has the structure of formula III:

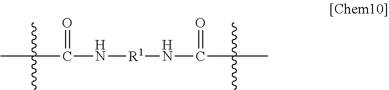

where R1 is as previously defined, and wherein the units A and B are arranged according to a linear, branched or cyclic, and preferably linear configuration.

The polymer may also comprise branching or grafting points in the polyorganosiloxane wherein one or more R groups in formula I or II is a group IV such that:

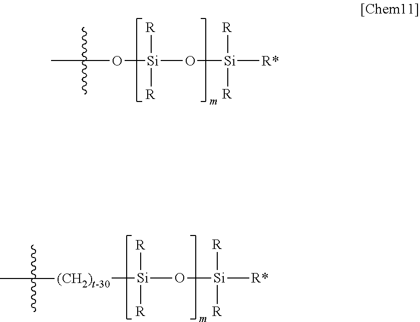

wherein R is as defined for formula I, and R* may represent a -L-O— group further coupling the side chain of a unit B of formula III, which may in turn be further coupled to the unit A of formula II, and so on, or else R* can represent -L-OH, a group R as previously defined, or a terminal group.

When the polyorganosiloxane includes such branching or grafting points, they may be present as T- or Q-type grafts, where T indicates that only one group R on the Si atom is a polyorganosiloxane chain, as indicated above and Q indicates that both groups R are polyorganosiloxanes.

These types of polyorganosiloxane compounds are called copolymer of silicone/polyurethane polymer and of branched or grafted T-resin or Q-resin.

Silicone/polyurethane polymers can also be prepared from functionalized isocyanate prepolymers. For example, an isocyanate prepolymer can be a difunctional or multi-functional polyorganosiloxane isocyanate, such as the polyorganosiloxane diisocyanate shown below in Formula V:

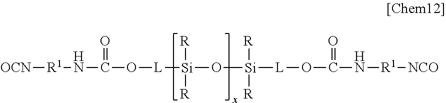

wherein R, R¹ and L are as defined previously, and wherein x is an integer between 0 and 5000, preferably between 1 and 200, more preferably between 10 and 100, and more preferably still between 10 and 50.

The prepolymer can also be multifunctional by introducing additional isocyanate groups carrying one or more groups R. The functionalized isocyanate prepolymer is reacted with a polyorganosiloxane functionalized by hydroxyl groups to obtain a compound of formula I or a multifunctional analogue thereof.

The prepolymer according to formula V generally has a molecular weight ranging from 4000 to about 15 000 Daltons. The prepolymer according to formulas I, Ia, Ib and in turn typically has a molecular weight ranging from 250 to about 15 000 Daltons.

In one embodiment of the invention, the silicone/polyurethane polymer is free or substantially free of polyalkylene glycol subunits, in particular polyethylene glycol (PEG) or polypropylene glycol (PPG).

By “essentially free of”, it is meant that the polymer comprises less than about 1% by weight, preferably less than about 0.5% by weight, and more preferably less than about 0.1% by 20% by weight of polyalkylene glycol subunits.

In a preferred embodiment, the silicone/polyurethane polymer used in the cosmetic compositions of the invention is a linear polymer comprising the reaction product of formula Ib with a diisocyanate selected from the group consisting of the diisocyanate of 1,6-hexamethylene, dicyclohexylmethane 4,4′-diisocyanate, isophorone diisocyanate, and combinations thereof.

Such a silicone/polyurethane polymer is for example available from Siltech Corporation in the form of a premix in isododecane, under the commercial reference SILMER UR 5050 or UR 100100 (45% in dry material of polymer in the isododecane).

According to another embodiment, a silicone/polyurethane polymer is used in the form of a premix in a hemi-squalane.

According to another embodiment, a silicone/polyurethane polymer is used in the form of a premix in a linear C9-C16 alkane or a mixture of linear C9-C16 alkanes, preferably a mixture of n-nonane and n-dodecane (C9-C12, VEGELIGHT SILK) or a mixture of n-undecane and n-tridecane (C11-C13, CETIOL ULTIMATE).

The silicone/polyurethane polymer according to the invention will be present in the composition of the invention in a content of at least 17.5% by weight of active material (dry material) of polymer. The content of silicone/polyurethane polymer in the composition of the invention will generally be less than 40%, or even less than or equal to 30% by weight of active material relative to the total weight of the composition. According to a particular embodiment, the silicone/polyurethane polymer will be present in a content ranging from 17.5 to 25%, in particular from 18 to 22% by weight of active material relative to the total weight of the composition.

According to a particular and preferred embodiment, the composition of the invention comprises less than 5% by weight of silicone resin, in particular less than 1% by weight of silicone resin, and preferably is free of silicone resin.

The term “silicone resin” is understood to mean in particular silicone resins of the MQ type, of the T type or of the MQT type, which are generally in solid form, in particular in powder form.

### Oil Phase

The oily phase of the composition of the invention comprises at least one or more linear or branched C8-C19 alkanes

As linear or branched C8-C19 alkanes, mention may be made in particular of:
C8-C16 isoalkanes (also called isoparaffins) such as isododecane, isodecane, isohexadecane, hemi-squalane, and for example the oils sold under the trade names ISOPAR® or PERMETYL®,
linear alkanes having respectively hydrocarbon chains in
   C9-C17, C10-C14, such as a mixture of undecane and tridecane, marketed by BASF Care Creations under the name Cetiol® Ultimate,
   C15-C19, such as those marketed by Seppic under the name EMOGREEN® L15,
   C9-C12, C12-C14, such as those marketed by BIOSYNTHIS under the name VEGELIGHT® SILK (INCI name C9-12 ALKANE), VEGELIGHT® 1214LC,
   n-dodecane (C12) and n-tetradecane (C14), in particular sold by Sasol respectively under the references PARAFOL® 12-97 and PARAFOL® 14-97,
   and mixtures thereof.

Thus, according to a particular embodiment, the composition comprises a C8-C19 alkane is selected from the group consisting of branched alkanes such as isododecane, isodecane, isohexadecane, hemi-squalane, linear C8-C19 alkanes such as nonane, dodecane, undecane, tridecane, n-tetradecane, n-pentadecane, n-nonadecane and mixtures thereof, in particular mixtures of linear alkanes C11/C13, C10/C12, C10/C14, and C15/C19.

According to a particular embodiment, the composition comprises at least isododecane as branched C8-C19 alkane.

According to a particular embodiment, the total content of linear or branched C8-C19 alkanes in the composition of the invention, in particular of isododecane, will be at least 30% by weight relative to the total weight of the composition. In particular, it will in particular range from 40 to 80% by weight, in particular from 50 to 70% by weight relative to the total weight of the composition.

The fatty phase of the composition of the invention is generally textured or structured by a gelling and/or texturizing agent, in particular a lipophilic gelling agent, a wax, a pasty fatty substance, or mixtures thereof.

According to a particular embodiment, the composition of the invention comprises at least one lipophilic gelling agent.

### Gelling Agents

As lipophilic gelling agent, mention may in particular be made of mineral gelling agents such as bentones dispersed in an oil. Mention can for example be made of the reference BENTONE GEL ISD V from Elementis with the INCI name Isododecane (and) Disteardimonium Hectorite (and) Propylene Carbonate.

Thus, according to a particular embodiment, the composition of the invention further comprises a lipophilic gelling agent, in particular a mineral gelling agent such as bentones, preferably a quaternary hectorite.

The total content of lipophilic gelling agent(s), in particular bentone, may range from 0.5 to 5%, in particular from 2 to 4% by weight relative to the total weight of the composition.

### Silica Powder

The composition further comprises at least one silica powder, preferably a hydrophobic treated silica powder.

The Applicant has indeed shown that the use of a silica powder allowed to formulate in a composition according to the invention, a very high dry material content of silicone/polyurethane polymer, greater than 16%, despite the prejudices taught in application FR3052357. It also brings comfort to the composition and the matte effect sought for applying make-up, in particular on the lips. As silica powders, mention may be made in particular of the following commercial references:

TABLE 1

| Fillers (commercial | | Oil intake | Water intake |
|---|---|---|---|
| reference) | INCI name | (mL/100 g) | (mL/100 g) |
| AMILON | SILICA AND | 130 | 145 |
| | AMINO ACID | | |
| MK-7 | SILICA | 245 | 260 |
| SPHERICA BA-1 | SILICA | 150 | 265 |

According to a particular embodiment, the silica powder is hydrophobic treated.

By ‘hydrophobic treated silica powder’, it is meant that the silica powder is surface treated with a lipophilic or hydrophobic agent. Mention may in particular be made of lipophilic agents selected from the group consisting of N-acylated amino acids or salts thereof, silicone agents, metallic soaps, lecithin and derivatives thereof, waxes, fatty esters and phospholipids.

Preferably, the silica powder is treated with a lauroyl lysine. Mention may be made in particular of the commercial reference AMILON® from the company IKEDA.

According to a particular embodiment, the silica powder, in particular the hydrophobic treated silica powder, is present in the composition in a content ranging from 2 to 10%, in particular from 3 to 8% by weight relative to the total weight of the composition.

According to a particular embodiment, the composition of the invention is an anhydrous liquid composition comprising:
a) A silicone/polyurethane polymer with an active material content ranging from 17.5 to 25% by weight relative to the total weight of the composition,
b) Isododecane,
c) A silica powder, preferably a hydrophobic treated powder, in a content ranging from 2 to 10% by weight relative to the total weight of the composition,
d) Optionally pigments, and
e) A mineral lipophilic gelling agent such as bentones.

Preferably in this embodiment, the powder is treated with a lauroyl lysine.

According to a particular embodiment, the composition of the invention is an anhydrous liquid composition comprising:
a) A silicone/polyurethane polymer with an active material content ranging from 17.5 to 25%, preferably 18 to 22% by weight relative to the total weight of the composition,
b) One or more linear or branched C8-C19 alkanes, in particular isododecane, in a content ranging from 40 to 80%, preferably 50 to 70% by weight relative to the total weight of the composition,
c) A silica powder, preferably a hydrophobic treated powder, in a content ranging from 2 to 10%, preferably 3 to 8% by weight relative to the total weight of the composition,
d) Optionally pigments, and
e) A mineral lipophilic gelling agent such as bentones, in a content ranging from 0.5 to 5%, preferably 2 to 4% by weight relative to the total weight of the composition.

According to a particular embodiment, the composition of the invention is an anhydrous liquid composition comprising:
a) A silicone/polyurethane polymer with an active material content ranging from 18 to 22% by weight relative to the total weight of the composition,
b) Isododecane, in a content ranging from 50 to 70% by weight relative to the total weight of the composition,
c) A hydrophobic treated silica powder, preferably with a lauroyl lysine, in a content ranging from 3 to 8% by weight relative to the total weight of the composition,
d) optionally pigments, and
e) a bentone, in a content ranging from 2 to 4% by weight relative to the total weight of the composition.

The composition may further comprise other additional fillers, provided that they do not impact the result obtained with the silica powder.

Within the meaning of the invention, by “fillers” is meant particles of any shape, colorless or white, of mineral or organic, natural or synthetic nature, which are in a (platelet, spherical or oblong) form which is insoluble and dispersed in the middle of the composition. The fillers are selected in particular from micas, of natural or synthetic origin, kaolin, zinc and titanium oxides; calcium carbonate, magnesium carbonate and hydrocarbonate; zinc, magnesium or lithium stearate, zinc laurate, magnesium myristate; synthetic polymer powders, such as polyethylene, polyesters, polyamides (for example nylon); polyacrylic or polymethacrylic acid powders, silicone powders; cellulose powders; glass and ceramic beads; powders of organic materials of natural origin such as crosslinked or non-crosslinked corn, wheat, rice, cotton starches, and mixtures thereof.

According to a particular embodiment, the composition of the invention further comprises at least one other filler, in particular selected from cellulose powders, cotton powders, mica and mixtures thereof.

According to a particular embodiment, the total content of filler(s) in the composition will in particular range from 2 to 10%, in particular from 3 to 8% by weight relative to the total weight of the composition.

A composition of the invention is in particular a make-up composition comprising at least one coloring material.

### Coloring Materials

By coloring material is meant within the meaning of the present invention, a compound capable of producing a colored optical effect when it is formulated in sufficient amount in an appropriate cosmetic medium.

A coloring material can be selected from organic or inorganic coloring materials, optical effect materials, and mixtures thereof, soluble or insoluble in the continuous phase of the invention, in this case the oily phase of the invention.

According to a particular embodiment, the coloring material(s) are selected in particular from mineral pigments, organic pigments, and mixtures thereof.

By “pigments” is meant white or colored, mineral or organic particles, insoluble in an aqueous solution, intended to color and/or opacify the resulting deposit. Mention may be made of inorganic pigments, organic pigments, and composite pigments (that is to say pigments based on inorganic and/or organic materials).

Among the “mineral pigments”, mention may be made, by way of examples, of titanium dioxide (rutile or anatase), optionally surface-treated; black, yellow, red and brown iron oxides; manganese violet; ultramarine blue chromium oxide hydrated chromium oxide and ferric blue.

For compositions intended for the lips, mention may be made, by way of examples, of titanium dioxide; black, yellow, red and brown iron oxides and manganese violet.

Among the “organic pigments”, mention may be made, for example, of the pigments D & C red no 19; D & C red no 9; D&C Red no 22; D&C Red no 21; D&C Red no 28; D&C Yellow no 6; D & C orange no 4; D & C orange no 5; D & C Red no 27; D & C red no 13; D & C Red no 7; D & C Red no 6; D & C Yellow no 5; D & C Red no 36; D & C Red no 33; D & C orange no 10; D & C yellow no 6; D & C Red no 30; D & C red no 3; D & C Blue 1; carbon black and lacquers.

In particular, the coloring material(s) are present in the composition in a content ranging from 2% to 20% by weight, preferably from 3% to 15% by weight, relative to the total weight of the composition.

### Galenic

The composition of the invention is a composition for make-up and/or care of keratinous materials, in particular a composition for make-up and/or care of the skin and/or the lips, eyelids or eyebrows, preferably the lips.

According to a first embodiment, the composition of the invention is a “top-coat” composition, which can be used on any make-up composition for the lips, to provide a long-lasting matte result (without transfer).

According to another embodiment, the composition of the invention is a make-up composition in a single step, to provide color, matte effect and hold (transfer resistance).

The composition of the invention is advantageously a liquid composition, in particular a liquid composition preferably for the lips.

In one embodiment of the invention, it can be in liquid form and be a fluid foundation, eyeshadow or fluid lipstick, eyeliner, sunscreen or skin coloring product.

Preferably, the composition is a make-up composition for the lips, in particular in the form of a gloss for the lips.

### Cosmetic Method

The invention also relates to a cosmetic method for caring for and/or making up keratinous materials comprising the application, to said keratinous materials, in particular the skin and/or the lips, preferably the lips, of a composition according to the invention.

The composition applied to the lips is preferably a liquid make-up composition for the lips, in particular a fluid lipstick.

The method according to the invention is in particular a method for making up the lips, intended to deposit on said lips a matte film on the lips, like a second skin.

The invention will now be illustrated in the following non-limiting examples. The % are expressed as % by weight of ingredient relative to the total weight of the composition, unless otherwise indicated.

### EXAMPLES

### Example 1 Fluid and Matte Lipstick: Selection of the Polymer Content and Nature of the Filler

In the present example, the composition according to the invention is compared with two compositions which comprise, for one, a lower content of polyurethane silicone polymer, and for the other, a cellulose powder instead of a silica powder.

SILMER® UR-5050 comprises Bis-hydroxypropyl dimethicone/SMDI copolymer at 45% by weight of dry material in isododecane.

AMILON: Silica (and) Lauroyl lysine (95/5) The composition of the invention and comparative example 2 in the table below each comprise 40% by weight of the commercial product SILMER® UR-5050 which itself consists of a polyurethane silicone polymer in isododecane according to a weight ratio (polymer/isododecane) of 45/55.

The formula of comparative example 1 comprises 36% by weight of the commercial product SILMER® UR-5050 described above (that is to say 16.2% by weight of dry material of polymer in the formula).

The formulas are shown in Table 2 below.

TABLE 2

| | Example of | Comparison 1 | |
|---|---|---|---|
| | the | (rate <17.5% | Comparison 2 |
| | invention | DM of polymer) | (other filler) |
| Ingredient (INCI name) | % by weight | | |
| SILMER ® UR-5050 | | | |
| Bis-hydroxypropyl | | | |
| dimethicone/SMDI copolymer | 18 | 16.2 | 18 |
| Isododecane | 22 | 19.8 | 22 |
| AMILON ® | | | |
| Silica (and) Lauroyl lysine (95/5) | 5 | 5 | — |
| CELLULOSE SILK COTTON | | | |
| Cellulose | — | — | 5 |
| BENTONE ® GEL | | | |
| Disteardimonium hectorite | 2.5 | | |
| Propylene carbonate | 0.75 | | |
| Isododecane | 21.75 | | |
| Organic pigments (lacquers) | 6.15 | | |
| Iron oxide | 0.1 | | |
| Isododecane | QSP 100 | | |
| Properties | | | |
| Stability of the composition at 8 h | ++ | + | −− |
| Transfer resistance | ++ | − | Not tested |

++ very satisfying
+ satisfying
− bad
−− very poor

The composition is prepared according to the following protocol:

The pigments of the formulas are passed through a three-cylinder in isododecane to obtain a homogeneous and fine dispersion of pigments.

The SILMER UR-5050 and the BENTONE GEL are mixed then gradually added to the dispersion of pigments from the previous step. Allow to homogenize with Rayneri, 5 min at 1500 rpm.

The filler is added and the mixture is allowed to homogenize with Rayneri for 10-15 minutes.

The liquid composition is applied to the lips.

Its properties of stability of the composition at 8 h and of transfer resistance are evaluated:
the stability of the formula is evaluated after 8 hours at rest at room temperature;
the transfer resistance property is evaluated as follows: the colored film is applied to the lips and after drying, a tissue is applied to the lips and the presence or absence of transferred color is observed.

The formula of example 1 (invention) forms a comfortable, homogeneous, and matte deposit with good lasting of the matte effect over time. The deposit is like a second skin.

On the other hand, the comparative formula 1 comprising a content of silicone polyurethane polymer of less than 17.5% in dry material in the above formula, has poor transfer resistance properties. The film partially transfers onto a support and does not solve the technical problem underlying the invention.

Comparative formula 2 is not stable after 8 hours at rest at room temperature. A change in texture results in significant de-mixing of the pigments and the appearance of two visible phases. Given this result, the transfer resistance property is not evaluated.

These results show that the deposition performance of a transfer resistant matte film is obtained thanks to the combination of a polymer content greater than or equal to 17.5% DM relative to the total weight of the composition and a powder of silica.

### Example 2: Evaluation of Lasting and Transfer Resistance Properties

Formula 1 of the example above (invention) was evaluated compared to a long-wearing matte liquid lipstick on the market, the Cream Lip Stain product from Sephora, which is marketed as a liquid lipstick which is ultra-matte, ultra-covering, long-wearing, without transfer with an irreproachable comfort. This product in particular comprises a silicone resin, oils and fillers. The reference product (“Control”) CREAM LIP STAIN from Sephora has the following list of ingredients: isododecane, dimethicone, trimethylsiloxysilicate, polybutene, petrolatum, cyclohexasiloxane, kaolin, disteardimonium hectorite, cera alba (beeswax), silica dimethyl silylate, aroma (flavor), glyceryl behenate/eicosadioate, propylene carbonate, Persea gratissima (avocado) oil, tocopherol, cyclopentasiloxane, benzyl alcohol, +/− may contain (mica, CI 15850 (red 7 lake), CI 19140 (yellow 5 lake), CI 77891 (titanium dioxide), CI 77491 (iron oxides), CI 77492 (iron oxides), CI 77499 (iron oxides), CI 15850 (red 6), CI 42090 (blue 1 lake), CI 75470 (carmine)).

### Lasting Test

Use is made of a Visia CR camera to take pictures of the mouth, from the front, eyes closed.

Lasting was visualized every two hours: from 6 a.m. to 6 p.m. after application. The composition of the invention offers a more matte effect with a more homogeneous film lasting.

The lasting of the composition of the invention over time is much greater than that of the Control. Indeed, the lasting of the control decreases after a few hours whereas that of the invention is maintained up to 18 hours after application.

The composition of the invention forms a soft and comfortable film.

### Transfer Resistance Test (Frictiometer)

To be “transfer resistant”, a product must withstand two types of stresses, static and dynamic friction.

To measure performance in this area, a Probe Frictiometer FR700 is used. The apparatus has a friction head, formed of a Teflon disk on which a disk is fixed (diameter 16 mm, thickness 1 mm).

A homogeneous film of the composition is applied to the volunteer's forearm. Once applied to the skin in contact with the dry film, the friction head is rotated at a constant speed (100 rpm for 10 seconds).

Lasting properties (transfer resistance) are evaluated by measuring the amount of product transferred onto the disc. For this purpose, a photo is taken that is subjected to image analysis. The zones on which the product is found are identified, quantified in number of pixels relative to the maximum contact zone of the disc on the zone of application of the formula.

At the end of the test, the composition of the invention has a very small amount of product having transferred to the disc (2% transfer), significantly lower than that having transferred for the control formula (34% transfer).

### Panel Testing

The composition of the invention and the control (Cream Lip Stain from Sephora) described above in the example were tested by a panel of volunteers.

Each volunteer applied one of the two formulas to their lips, and kept it on throughout the day.

The panel volunteers then answered a questionnaire aimed at characterizing the properties of the formula tested (composition according to the invention or Control), in terms of transfer resistance (at T0 and T12h) and comfort during the day (at T12h).

The results are shown in the tables below, the first corresponding to T0 (application of the formula), and the second being carried out at the end of the test day.

Evaluation at T0 (after Application and Drying of the Formula on the Lips)

TABLE 3

| | | Example according | |
|---|---|---|---|
| | | to the invention | Comparative control |
| Number of volunteers (n) | | 17 | 10 |
| | | % | |
| Transfer | Transfers little | 47 | 80 |
| resistance | or a little | | |
| of the | Does not | 53 | 20 |
| color | transfer at all | | |

### Evaluation at T+12 Hours

TABLE 4

| | | Example | |
|---|---|---|---|
| | | according | |
| | | to the | |
| | | invention | Control |
| Number of volunteers (n) | | 17 | 10 |
| | | % | |
| Comfort | Moderately comfortable | 35 | 80 |
| | Very comfortable | 65 | 20 |
| Lasting of the color | 6 hours or less | 47 | 100 |
| (homogeneity) | 8 hours | 53 | 0 |
| Transfer resistance | Transfers little or a little | 53 | 100 |
| of the color | Does not transfer at all | 47 | 0 |

These results show that the composition of the invention has properties superior to those of the Control, in terms of comfort, color lasting (homogeneity) and transfer resistance, both upon application (TO) and after one day of use.

These performances allow to preserve the product for a longer period and preserve its integrity (homogeneity and color) during the day.

### Example 2 “Top Coat” Formula

An uncolored composition (absent of pigments) according to the invention is prepared.

The formula is shown in the table below.

TABLE 5

| | Example of the invention |
|---|---|
| Ingredient (INCI name) | % by weight |
| SILMER ® UR-5050 | |
| Bis-hydroxypropyl dimethicone/SMDI | 40 |
| copolymer | |
| Isododecane | |
| AMILON ® | |
| Silica (and) Lauroyl lysine (95/5) | 5 |
| BENTONE ® GEL | |
| Disteardimonium hectorite | 25 |
| Propylene carbonate | |
| Isododecane | |
| Isododecane | 30 |

SILMER ® UR-5050 comprises Bis-hydroxypropyl dimethicone/SMDI copolymer at 45% by weight of dry material in isododecane.
AMILON: Silica (and) Lauroyl lysine (95/5)

The composition is prepared according to the following protocol:

The composition of example 2 is applied to the lips to form a film. It can advantageously be used as a Top Coat, after the application of a first layer of a colored film on the lips. It provides a matte make-up result with improved lasting (transfer resistance).

1.-13. (canceled)

14. A cosmetic composition for care and/or make-up of keratinous materials, comprising, in a physiologically acceptable medium:
a) a silicone/polyurethane polymer with an active material content of at least 17.5% by weight, relative to the total weight of the composition,
b) one or more linear or branched C8-C19 alkanes, and
c) a silica powder.

15. The composition according to claim 14, wherein further comprising pigments.

16. The composition according to claim 14, wherein the composition is a liquid and anhydrous composition.

17. The composition according to claim 14, further comprising a lipophilic gelling agent.

18. The composition according to claim 14, wherein the composition comprises less than 5% by weight of silicone resin.

19. The composition according to claim 14, wherein the silicone/polyurethane polymer is the reaction product of one polyorganopolysiloxane functionalized with hydroxyl groups, corresponding to the structure of formula (I) 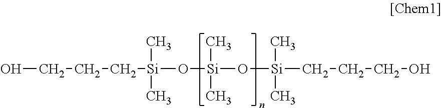
wherein n is an integer between 0 and 5000,
with a diisocyanate compound.

20. The composition according to claim 14, wherein the silicone polyurethane polymer is present in a content ranging from 17.5 to 25%, by weight of active material relative to the total weight of the composition.

21. The composition according to claim 14, wherein the C8-C19 alkane is selected from the group consisting of branched alkanes, linear C8-C19 alkanes and mixtures thereof.

22. The composition according to claim 14, wherein the composition comprises at least isododecane.

23. The composition according to claim 14, wherein the silica powder is hydrophobic treated.

24. The composition according to claim 14, wherein the composition comprises a silica powder content ranging from 3 to 8% relative to the total weight of the composition.

25. The composition according to claim 16, wherein the composition comprises:
a) a silicone/polyurethane polymer with an active material content ranging from 17.5 to 25% by weight relative to the total weight of the composition,
b) isododecane
c) a silica powder, in a content ranging from 2 to 10% by weight relative to the total weight of the composition,
d) optionally pigments, and
e) a mineral lipophilic gelling agent.

26. The composition according to claim 14, further comprising at least one other filler.

27. A cosmetic method for caring for and/or making up keratinous materials, comprising the application to said keratinous materials, of a composition according to claim 14.

28. The cosmetic method according to claim 27, wherein the keratinous materials are the lips.
